(19) United States
(12) Patent Application Publication
(10) Pub. No.: US 2023/0115287 A1
(43) Pub. Date: Apr. 13, 2023

(54) Disinfection Devices and Methods

(71) Applicant: Autonomous Units ApS, Langeskov (DK)

(72) Inventor: Flemming KJÆR THINGGAARD, Langeskov (DK)

(21) Appl. No.: 18/075,599

(22) Filed: Dec. 6, 2022

Related U.S. Application Data
(63) Continuation of application No. PCT/EP2021/065417, filed on Jun. 9, 2021.

(30) Foreign Application Priority Data
Jun. 11, 2020 (DK) PA 2020 00682
Apr. 28, 2021 (DK) PA 2021 00425

(51) Int. Cl. A61L 2/24; A61L 2/22

(52) U.S. Cl. CPC A61L 2/24; A61L 2/22; A61L 2202/14; A61L 2202/16; A61L 2202/25

(57) ABSTRACT

A disinfection unit configured to be operated in a room having a ceiling includes a detection unit arranged and configured to detect a height of the room and one or more nozzles each being arranged to generate a jet and spray a disinfection fluid into the room. The disinfection unit includes a control unit configured to calculate a quantity of disinfection fluid to be sprayed into the room on the basis of a predefined required degree of purity.

### CROSS-REFERENCE TO RELATED APPLICATIONS

This application is a continuation under 35 U.S.C. 111 of International Patent Application No. PCT/EP2021/065417, filed Jun. 9, 2021, which claims the benefit of and priority to Danish Application Nos. PA 2020 00682, filed Jun. 11, 2020, and PA 2021 00425, filed Apr. 28, 2021, each of which is hereby incorporated by reference in its entirety.

### FIELD OF INVENTION

The present invention relates to a disinfection device and a method for disinfecting one or more rooms of a building.

### BACKGROUND

US20160271803A1 discloses a robot platform for remotely controlled and autonomous disinfection of a facility. The robot platform comprises a mobile unit configured to move the robot platform. The robot platform also comprises a disinfection module having a plurality of ultraviolet radiation emitters disposed above the drive mechanism. The robot platform, however, is not capable of sufficiently effectively disinfecting building rooms because the ultraviolet radiation will not reach all surfaces.

US2003030398A1 discloses a robot system for performing a function in an area, the area having an area layout including at least one area segment. In one embodiment, the robot system comprises a disinfectant applicator device configured to disinfect the area. The robot system, however, is not capable of disinfecting the area autonomously because not all area-specific parameters (including room dimensions) are detected by the robot system.

Thus, there is a need for a disinfection device and a method for disinfecting one or more rooms of a building which reduces or even eliminates the above-mentioned disadvantages of the prior art.

### BRIEF DESCRIPTION

It is an object of the present systems to provide a disinfection device that is capable of sufficiently effectively disinfecting building rooms in an autonomous manner, and to provide a method for disinfecting one or more rooms of a building, wherein the method sufficiently effectively disinfects building rooms in an autonomous manner.

A disinfection unit according to the present disclosure is a disinfection unit configured to be operated in a room having a ceiling, wherein the disinfection unit comprises one or more nozzles each being arranged to generate a jet and hereby spray a disinfection fluid into the room, wherein the disinfection unit comprises a control unit configured to calculate a quantity of disinfection fluid to be sprayed into the room on the basis of a predefined required degree of purity, wherein the disinfection unit:

a) comprises a mobile device or

b) is configured to be electrically and mechanically connected to a mobile device,

wherein the mobile device is configured to move the disinfection unit, wherein the disinfection unit comprises a position determination device configured to determine the position of the disinfection unit, wherein the disinfection unit comprises a detection unit arranged and configured to detect a height of the room.

Hereby, it is possible to provide a disinfection device that is capable of sufficiently effectively disinfecting building rooms in an autonomous manner, while the prior art solutions require manual settings (e.g. that manual measurements are made and entered) before the disinfection device is operated.

A disinfection unit disclosed herein is configured to be operated in a room having a ceiling.

A disinfection unit disclosed herein comprises one or more nozzles each arranged to generate a jet and hereby spray a disinfection fluid into the room. The pressure for creating the jet may be generated by a pump or compressor.

In an embodiment, the speed of the pump or compressor is regulated by a regulator. In an embodiment, the regulator is a frequency converter. In an embodiment, the regulator is a pulse width modulation (PWM) controller. The PWM controller is configured to reducing the average power delivered by an electrical signal, by chopping it up into discrete parts. Hereby, the duty cycle can be changed.

In an embodiment, the control unit is configured to calculate a quantity of disinfection fluid to be sprayed into the room on the basis of a predefined required degree of purity and the volume of the room.

In an embodiment, the control unit is configured to calculate the estimated time for spraying the disinfection fluid into the room on the basis of a predefined required degree of purity and the volume of the room.

In an embodiment, the disinfection unit comprises a mobile device. This means than the disinfection unit has an integrated mobile device that is configured to move the disinfection unit.

In an embodiment, the disinfection unit is configured to be electrically and mechanically connected to a mobile device. In this embodiment, the mobile device is not integrated into the disinfection unit.

The disinfection unit comprises a position determination device configured to determine the position of the disinfection unit. In an embodiment, the position determination device is integrated in the disinfection unit.

In an embodiment, the position determination device is integrated in the mobile device. Hereby, the disinfection unit can benefit from using the determination device integrated in the mobile device.

The disinfection unit comprises a detection unit arranged and configured to detect at least one height of the room.

In an embodiment, the disinfection unit comprises a detection unit arranged and configured to detect all heights of the room.

The height detection may be done by the disinfection unit while the mobile device moves the disinfection unit within the room.

In an embodiment, the disinfection unit is configured to carry out a height detection while the mobile device is moved along a predefined path. The predefined path should be selected in such a manner that a sufficiently large number of height measurements are available in order to ensure that a predefined number of height measurements are available for square meter of the ceiling. In an embodiment, the predefined number of height measurements available is at least 1 for each square meter of the ceiling. In an embodiment, the predefined number of height measurements available is at least 2 for each square meter of the ceiling. In an embodiment, the predefined number of height measurements available is at least 1 for each two square meters of the ceiling. In an embodiment, the predefined number of height measurements available is at least 1 for each four square meters of the ceiling.

In an embodiment, the disinfection unit comprises a housing provided with a plurality of nozzles, wherein the nozzles are directed in different directions. In an embodiment, the directions of the corresponding jets are directed in different directions.

Hereby, it is possible to provide a distribution of the disinfection fluid.

In an embodiment, the disinfection unit is configured to carry out a position specific distribution of the disinfection fluid so that the disinfection fluid is distributed in dependency of position specific height measurements of the detection unit. Hereby, it is possible to take different height distributions of a room into account.

In an embodiment, the disinfection unit is configured to carry out a position specific distribution of the disinfection fluid in such a manner that the volume of the disinfection fluid is distributed in dependency of one or more height measurements carried out by the detection unit while the disinfection unit is within 2 meters from the actual position.

In an embodiment, the disinfection unit is configured to carry out a position specific distribution of the disinfection fluid in such a manner that the volume of the disinfection fluid is distributed in dependency of one or more height measurements carried out by the detection unit while the disinfection unit is within 1 meter from the actual position.

By the term “the actual position” is meant the horizontal coordinates of the disinfection unit in the building. “The actual position” would typically be defined by a X value and a Y value, wherein X and Y represent two orthogonal horizontal axes extending along the floor of the room.

In an embodiment, the disinfection unit is configured to distribute the disinfection fluid evenly in the space independently of the height.

In an embodiment, the nozzles are symmetrically arranged in such a manner that the jet of a central nozzle extends along the frontal plane of the disinfection unit.

In an embodiment, the jet of nozzles adjacent to the central nozzle are angled relative to the frontal plane of the disinfection unit.

In an embodiment, the angle α between the first adjacent nozzle and the jet of the central nozzle corresponds to the angle β between the second adjacent nozzle and the jet of the central nozzle.

In an embodiment, the control unit is arranged and configured to receive signals from one or more external devices, wherein the control unit is configured to control the activity of the disinfection unit on the basis of these signals.

Hereby, it is possible to take external conditions into consideration when operating the disinfection unit.

In an embodiment, the signals are wirelessly sent signals.

In an embodiment, the control unit is arranged and configured to receive signals from one or more external devices, wherein the control unit is configured to control the activity of the mobile device on the basis of these signals.

In an embodiment, the one or more external devices include a communication unit that receives signals from the ventilation system of the building, in which the disinfection unit is operated.

In an embodiment, the one or more external devices include a reader box arranged to detect when a member of staff leaves or enters the building, wherein the reader box comprises a key reader configured to read a key member used by a person to acknowledge that the person enters the building or leaves the building.

In an embodiment, the control unit is configured to stop the disinfection unit if the control unit receives a signal that indicates that a person is in the room to be disinfected.

In an embodiment, the control unit is configured to stop the disinfection unit if the control unit receives a signal that indicates that the ventilation system is turned on.

In an embodiment, the control unit is configured to stop the disinfection unit if the control unit receives a signal that a door is being opened. The signal may be transmitted by a sensor arranged to detect opening of the door.

In an embodiment, the control unit is configured to stop the disinfection unit if the control unit receives a signal that a person enters a room in the building, in which the disinfection is arranged. The signal may be transmitted by a motion sensor arranged to detect nearby motion. In an embodiment, the motion sensor is a passive infrared sensor (PIR sensor) that is configured to measure infrared (IR) light radiating from objects (such as a person) in its field of view.

In an embodiment, the disinfection system disclosed herein is a disinfection system that comprises:
a disinfection unit as described herein;
a ventilation system; and
one or more communication units arranged and configured to detect the activity of the ventilation system,
   wherein the disinfection system is configured to control the activity of the disinfection unit on the basis of the detected activity of the ventilation system.

Hereby, the disinfection system is capable of sufficiently effectively disinfecting building rooms in an autonomous manner and taking the activity of the ventilation system into consideration.

The one or more communication units is integrated in the ventilation system.

In an embodiment, the disinfection system comprises a reader box arranged and configured to read a key member used by persons entering or leaving the building in which the room is placed, wherein the communication unit is arranged and configured to detect the activity of a reader box, wherein the disinfection system is configured to control the activity of the disinfection unit on the basis of the detected activity of the reader box.

Hereby, it is possible to ensure that the disinfection unit is only operated when no persons are present in the room or in the building, in which the room is located.

By the term “activity of a reader box” is meant events detected by the activity of a reader box. Such events may include that a person acknowledges that he or she is leaving the building or is entering the building.

In an embodiment, the communication unit is integrated in the reader box. Accordingly, the reader box sends signals directly or indirectly (via an intermediate device) to the disinfection unit.

In an embodiment, the disinfection system is configured to stop the disinfection unit if it is detected that one or more of the ventilation devices are turned on. This can be done by applying a control algorithm that only allows the disinfection unit to be operated if it has been verified that no “turned on” signals are received from any of the ventilation devices.

In an embodiment, the disinfection system is configured to stop the disinfection unit if it is detected that a door is being opened. The signal may be transmitted by a sensor arranged to detect opening of the door.

In an embodiment, the disinfection system is configured to stop the disinfection unit if it is detected that a person enters a room in the building, in which the disinfection is arranged. The signal may be transmitted by a motion sensor arranged to detect nearby motion. In an embodiment, the motion sensor is a passive infrared sensor (PIR sensor) that is configured to measure infrared (IR) light radiating from objects (such as a person) in its field of view.

In an embodiment, a method for disinfecting a room having a ceiling uses a disinfection unit that comprises one or more nozzles each being arranged to generate a jet and hereby spray a disinfection fluid into the room, wherein the disinfection unit comprises a control unit configured to calculate a quantity of disinfection fluid to be sprayed into the room on the basis of a predefined required degree of purity, wherein the disinfection unit:

a) comprises a mobile device or

b) is configured to be electrically and mechanically connected to a mobile device,

wherein the mobile device is configured to move the disinfection unit, wherein the disinfection unit comprises a position determination device configured to determine the position of the disinfection unit, wherein the method comprises the step of detecting a height of the room using a detection unit of the disinfection unit.

Hereby, it is possible to provide a method that sufficiently effectively disinfects building rooms autonomously.

It may be an advantage that the method comprises the step of controlling the activity of the disinfection unit on the basis of signals received from one or more external devices.

In an embodiment, the step of controlling the activity of the disinfection unit is carried out on the basis of signals from the key system when the last person leaves the building, in which the room is placed.

In an embodiment, the step of controlling the activity of the disinfection unit is carried out on the basis of signals indicating the activity of a ventilation system. Hereby, it is possible to only operate the disinfection unit when the ventilation system is turned off.

In an embodiment, the step of controlling the activity of the disinfection unit is carried out on the basis of one or more signals indicating that a door is being opened. The signal may be transmitted by a sensor arranged to detect opening of the door.

In an embodiment, the step of controlling the activity of the disinfection unit is carried out on the basis of one or more signals indicating that a person enters a room in the building, in which the disinfection is arranged. The signal may be transmitted by a motion sensor arranged to detect nearby motion. In an embodiment, the motion sensor is a passive infrared sensor (PIR sensor) that is configured to measure infrared (IR) light radiating from objects (such as a person) in its field of view.

### BRIEF DESCRIPTION OF THE DRAWINGS

The present inventions will become more fully understood from the detailed description given herein below. The accompanying drawings are given by way of illustration only, and thus, they are not limitative of the present inventions. In the accompanying drawings:

FIG. 1A shows a side view of a disinfection unit according to an embodiment;

FIG. 1B shows a front view of the disinfection unit shown in FIG. 1A;

FIG. 2A shows a perspective top view of the disinfection unit shown in FIG. 1A and in FIG. 1B;

FIG. 2B shows a top view of the disinfection unit shown in FIG. 1A and in FIG. 1B;

FIG. 3A shows a disinfection unit being operated in a first location;

FIG. 3B shows a disinfection unit being operated in a second location;

FIG. 4 shows a schematic view of a disinfection system according to an embodiment;

FIG. 5A shows two-dimensional sensor data detected by sensors of a disinfection unit according to an embodiment in a first room;

FIG. 5B shows two-dimensional sensor data detected by sensors of a disinfection unit according to an embodiment in a second room;

FIG. 6 shows a disinfection unit according to an embodiment operated in a room;

FIG. 7A shows a top view of a disinfection unit according to an embodiment operated in a room; and

FIG. 7B shows a side view of a disinfection unit according to an embodiment operated in a room.

### DETAILED DESCRIPTION

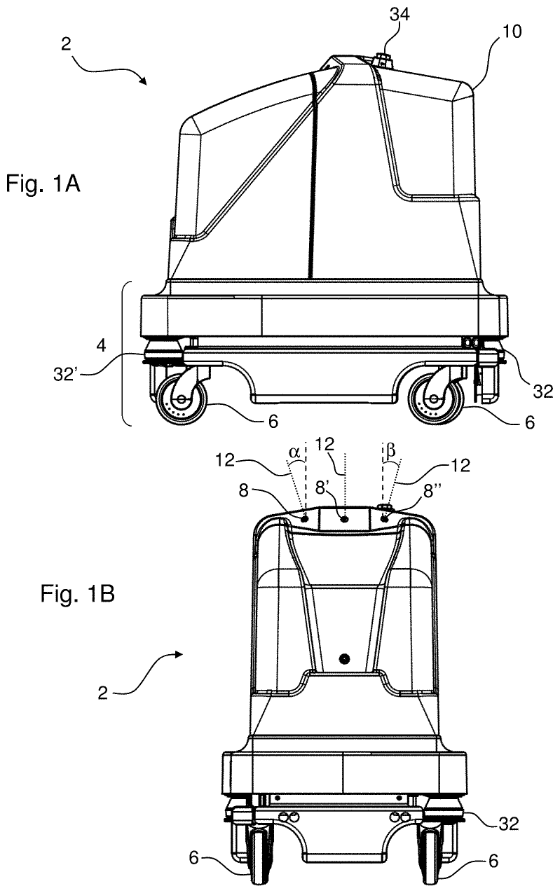

Referring now in detail to the drawings for the purpose of illustrating embodiments of the present inventions, a disinfection unit 2 is illustrated in FIG. 1A.

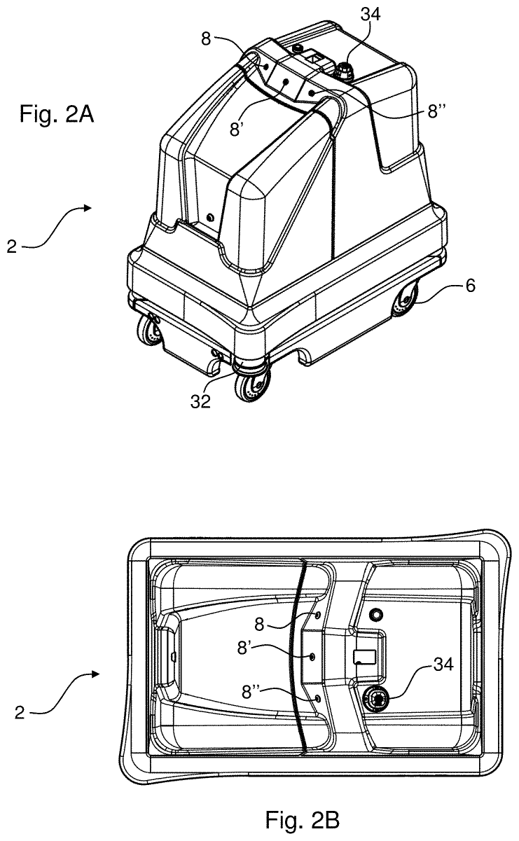

FIG. 1A illustrates a schematic side view of a disinfection unit 2 according to an embodiment. FIG. 1B illustrates a front view of the disinfection unit shown in FIG. 1A. FIG. 2A illustrates a perspective top view of the disinfection unit shown in FIG. 1A and in FIG. 1B. FIG. 2B illustrates a top view of the disinfection unit shown in FIG. 1A and in FIG. 1B.

The disinfection unit 2 comprises a housing 10 provided with a compartment for a disinfection fluid. A detection unit 34 is arranged at the upper portion of the housing 10. The detection unit 34 is arranged and configured to detect the height H₁, H₂, H₃ of the room, in which the disinfection unit 2 is operated. In an embodiment, the detection unit 34 is a laser distance sensor. A laser distance sensor ensures high process stability. In an embodiment, the detection unit 34 is an ultrasonic distance sensor.

The disinfection unit 2 comprises three nozzles 8, 8′, 8″ arranged at the upper portion of the housing 10. Each nozzle 8, 8′, 8″ is arranged to generate a jet 12 and hereby spray a disinfection fluid into the room in which the disinfection unit 2 is operated.

In FIG. 1B it can be seen that the central nozzles 8, 8′, 8″ are symmetrically arranged. The jet of the central nozzle 8′ extends along the frontal plane of the disinfection unit 2. The jet 12 of nozzles 8, 8″ adjacent to the central nozzle 8′ are angled relative to the frontal plane of the disinfection unit 2. It can be seen that the angle α between the first nozzle 8 and the jet 12 of the central nozzle 8′ corresponds to the angle θ between the third nozzle 8″ and the jet 12 of the central nozzle 8′.

The disinfection unit 2 is arranged on a mobile device 4 that comprises a plurality of ground engaging wheels 6. The mobile device 4 comprises sensors 32, 32′ configured to detect any objects present in the room, in which the disinfection unit 2 is operated. In an embodiment, the sensors 32, 32′ are laser sensors.

The disinfection unit 2 comprises a control unit that is arranged and configured to receive and process data. The disinfection unit 2 is configured to be mechanically and electrically connected to the mobile device 4. Hereby, it is possible to send sensor data detected by the mobile device 4 to the control unit. Accordingly, the disinfection unit 2 can process these sensor data and use them to calculate the volume of the room in which the disinfection unit 2 is operated.

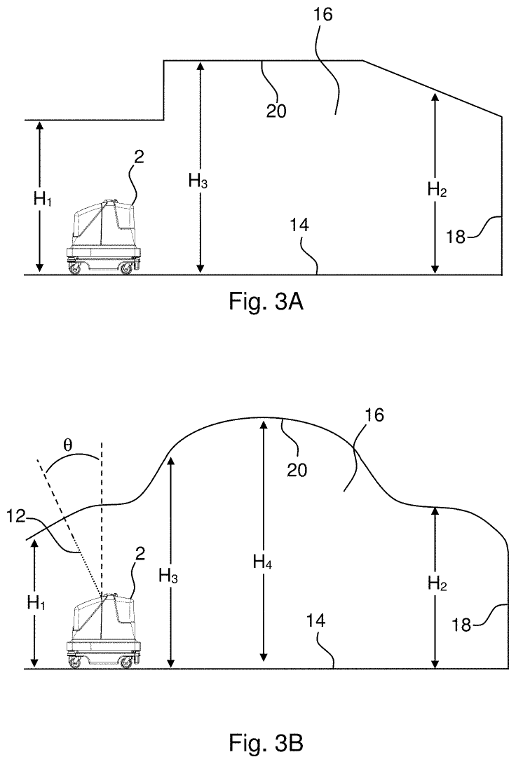

FIG. 3A illustrates a disinfection unit 2 according to an embodiment being operated in a room 16. The room 16 comprises a floor 14, a wall 18 and a ceiling 20. The ceiling 20 comprises several different sections of different heights H₁, H₂, H₃.

The disinfection unit 2 is arranged in a first section of the room 16 having a first constant height H₁. This section is located next to a section having a larger constant height H₃. This section is located next to a section having a height H₂ decreasing from H₃ to H₁.

The detection unit 2 comprises a detection unit that is arranged and configured to detect the heights H₁, H₂, H₃ of the room 16. Accordingly, the disinfection unit 2 is configured to detect and determine the dimension of the room 16 so that the volume of the room 16 can be automatically determined.

FIG. 3B illustrates a disinfection unit 2 being operated in a second room 16 defined by a floor 14, a wall 18 as well as a ceiling 20 having a central arch arranged between side portions. It can be seen that the height varies. In the side portions the height H₁, H₂ is lower than the heights H₃, H₄ in the central arch.

The disinfection unit 2 comprises nozzles arranged and configured to spray jets 12 in an upwards direction. The angle θ between the spray directing and vertical is indicated. In an embodiment, the angle θ between the spray directing and vertical is 5-45 degrees.

In an embodiment, the angle θ between the spray directing and vertical is in a range of 10-20 degrees.

In an embodiment, the angle θ between the spray directing and vertical is in a range of 20-30 degrees.

In an embodiment, the angle θ between the spray directing and vertical is in a range of 30-40 degrees.

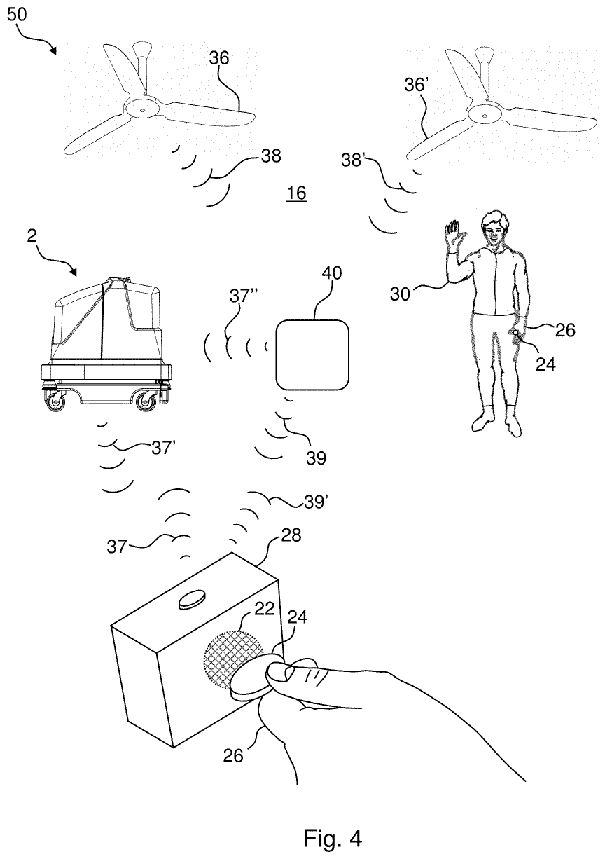

FIG. 4 illustrates a schematic view of a disinfection system 50 according to an embodiment. The disinfection system 50 comprises a disinfection unit 2 corresponding to the one shown in and explained with reference to the preceding figures.

The disinfection unit 2 is configured to carry out the most efficient disinfection and in the same time avoid exposing personnel to the disinfection fluid sprayed by the disinfection unit 2. This is achieved by detecting the activity of ventilation devices 36, 36′ installed in the room 16 to be disinfected by the disinfection unit 2. It should be noted that even though the ventilation devices 36, 36′ are illustrated as ceiling mounted fans, the ventilation devices 36, 36′ may be any type of ventilation device including ventilating devices arranged and configured to ventilate the room 16. Accordingly, the ventilation devices 36, 36′ may be arranged and configured to replace air from the room 16 with air from outside.

The disinfection system 50 comprises a communication unit 40 arranged and configured to detect the activity of ventilation devices 36, 36′. This is done by receiving signals 38, 38′ sent from the ventilation devices 36, 36′.

The communication unit 40 is also arranged and configured to detect the activity of a reader box 28. The reader box 28 is used by the staff 30 working in the building in which room 16 is placed. When a member of staff leaves or enters the building, a key member 24 is used to acknowledge that the person 30 enters the building or leaves the building. The reader box 28 comprises a key reader 22 integrated in or attached to the reader box 28. The reader box 28 is arranged and configured to send signals 37 to the disinfection unit 2 (e.g., to a control unit thereof). In an embodiment, the reader box 28 is arranged and configured to send signals 39′ to the communication unit 40, wherein the communication unit 40 is arranged and configured to transmit received signals 38′, 39′ to the disinfection unit 2 (e.g., to a control unit thereof).

In practice, the person 30 holds a key member 24 in his hand 26 and holds the key member 24 in close proximity to the key reader 22 of the reader box 28 in order to acknowledge entrance into and/or exit from the building, in which the room 16 is placed.

In an embodiment, the disinfection system 50 is configured to stop the disinfection unit 2 if it is detected that one or more of the ventilation devices 36, 36′ are turned on. This can be done by applying a control algorithm that only allows the disinfection unit 2 to be operated if it has been verified that no “turned on” signals are received from any of the ventilation devices 36, 36′.

In an embodiment, the disinfection system 50 is configured to stop the disinfection unit 2 if it is detected that one or more persons 30 are present in one or more predefined rooms 16 of the building. This can be done by applying a control algorithm that only allows the disinfection unit 2 to be operated if it has been verified that no persons 30 are present in one or more predefined rooms 16 of the building.

In an embodiment, the disinfection system 50 is configured to stop the disinfection unit 2 if it is detected that one or more persons 30 are present in the building. This can be done by applying a control algorithm that only allows the disinfection unit 2 to be operated if it has been verified that no persons 30 are present in the building.

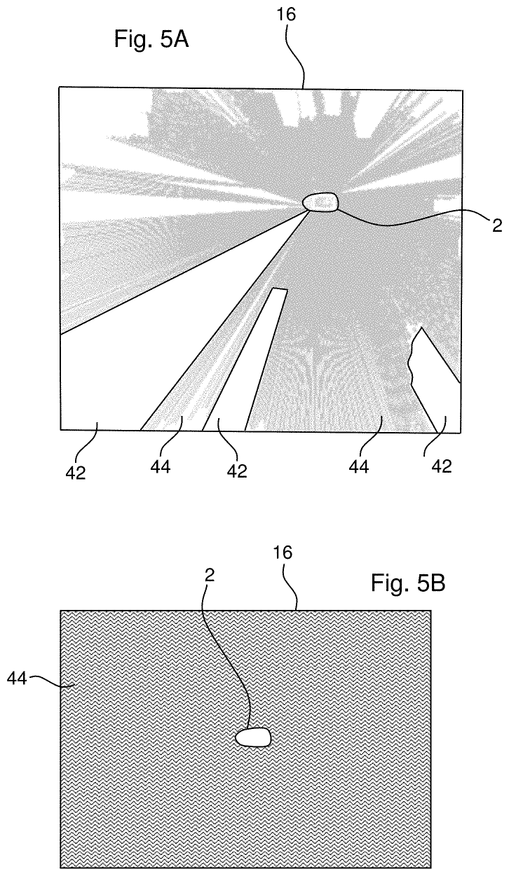

FIG. 5A illustrates two-dimensional sensor data of a room 16 detected by sensors of a disinfection unit according to an embodiment. The two-dimensional sensor data may be detected by sensors 32, 32′ as shown in and explained with reference to FIG. 1A, FIG. 1B and FIG. 2A.

It can be seen that the disinfection unit 2 is arranged close to the central part of the room 16. Moreover, it can be seen that the two-dimensional sensor data comprises non-visible areas 42 as well as visible areas 44. The non-visible areas 42 detected by the disinfection unit 2 may be caused by an object that provides shade.

FIG. 5B illustrates two-dimensional sensor data of another room 16 detected by sensors of a disinfection unit according to an embodiment. The two-dimensional sensor data may be detected by sensors 32, 32′ as shown in and explained with reference to FIG. 1A, FIG. 1B and FIG. 2A.

The disinfection unit 2 is arranged centrally in the room 16. Two-dimensional sensor data comprises no non-visible areas but only visible areas 44. This indicates that no objects are placed on the floor of the room 16.

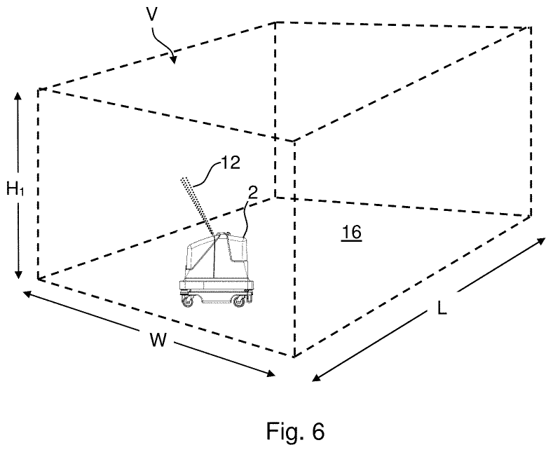

FIG. 6 illustrates a disinfection unit 2 according to an embodiment operated in a room 16. The room 16 has a rectangular floor having a length L and a width W. Accordingly, the area of the floor can be calculated as the product between the length L and the width W. The room 16 has a height H₁. Therefore, the disinfection unit 2 can automatically detect that the volume V of the room 16 is given by the product of the length L, the width W and the height H₁.

In practice, the disinfection unit 2 can initially automatically detect the dimensions of the room 16. Accordingly, the volume of the room can be calculated on the basis of the detected dimensions of the room 16.

When the volume of the room has been calculated, the disinfection unit 2 can automatically disinfect the room 16 in an efficient manner.

The disinfection unit 2 can, by means of the mobile device carrying it, move freely in the room 16. The control unit (not shown) of the disinfection unit 2 is configured to calculate the volume V of the room 16 on the basis of the data collected by the sensors and detection unit of the disinfection unit 2.

The control unit of the disinfection unit 2 is configured to calculate the disinfection time required by the disinfection unit 2 to obtain the desired degree of purity.

Accordingly, the disinfection unit 2 is configured to carry out the disinfection process in the shortest possible time and with the greatest possible precision. The process can be carried out automatically by the disinfection unit 2 without requiring any staff.

In an embodiment, the disinfection unit 2 is configured to select the pressure applied to create the jet 12 on the basis of measurements carried out by any of the sensors and detection unit of the disinfection unit 2.

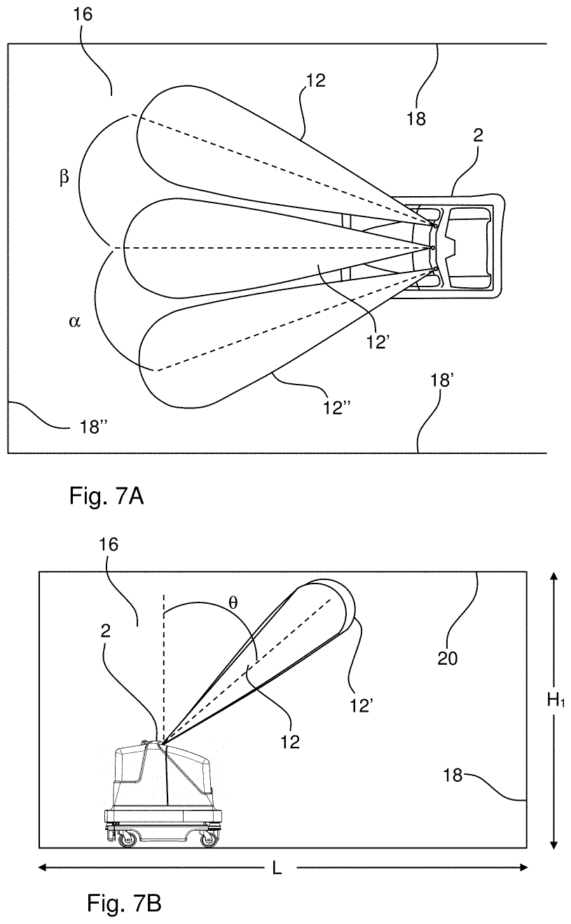

FIG. 7A illustrates a top view of a disinfection unit 2 according to an embodiment operated in a room 16. The room 16 has several walls 18, 18′, 18″ defining the boundaries of the room 16. It can be seen that the disinfection unit 2 comprises three nozzles each generating a jet 12, 12′, 12″. The directions of the jets 12, 12′, 12″ differ. The angle (viewed in the horizontal plane) α, β between adjacent jets 12, 12′, 12″ is approximately 20 degrees.

The disinfection unit 2 is configured to be maintained in a zone that ensures that the jets 12, 12′, 12″ do not spray directly on any of the walls 18, 18′, 18″. Hereby, it is possible to prevent the disinfection fluid from condensing on any wall surface.

FIG. 7B illustrates a side view of a disinfection unit 2 according to an embodiment operated in a room 16 having a wall 18 and a ceiling 20. The disinfection unit 2 comprises several nozzles each generating a jet 12, 12′. The angle θ of the jets 12, 12′ (viewed in the vertical plane) relative to vertical is approximately 70 degrees.

The disinfection unit 2 is configured to be maintained in a zone that ensures that the jets 12, 12′ do not spray directly on the ceiling 20. Hereby, it is possible to prevent the disinfection fluid from condensing on the ceiling 20.

The height H₁ of the ceiling is indicated. If the disinfection unit 2 is moved into a room, in which the height of the ceiling is different, the detection unit of the disinfection unit 2 will detect the height and adjust the pressure of the nozzles. Hereby, the disinfection unit 2 is capable of controlling the jets 12, 12′ in such a manner that no disinfection fluid will condense on the ceiling 20. The disinfection unit 2 comprises one or more other devices (e.g. sensors) capable of detecting the distance between the disinfection unit 2 and any adjacent wall 18. Accordingly, the disinfection unit 2 is capable of controlling the jets 12, 12′ in such a manner that no disinfection fluid will condense on any wall 18.

### LIST OF REFERENCE NUMERALS

2 Disinfection unit
4 Mobile device
6 Wheel
8, 8′, 8″ Nozzle
10 Housing
12, 12′, 12″ Jet
14 Floor
16 Room
18, 18′, 18″ Wall
20 Ceiling
22 Key reader
24 Key member
26 Hand
28 Reader box
30 Person
32, 32′ Sensor
34 Detection unit
36 Ventilation unit
37, 37′, 37″ Signal
38, 38′ Signal
39, 39′ Signal
40 Communication unit
42 Non-visible area
44 Visible area
50 Disinfection system
H₁, H₂ Height
H₃, H₄ Height
α, β, θ Angle
W Width
L Length
V Volume

What is claimed is:

1. A disinfection unit configured to be operated in a room having a ceiling, the disinfection unit comprising:
a position determination device configured to determine the position of the disinfection unit;
a detection unit arranged and configured to detect a height of the room;
a control unit configured to calculate a quantity of disinfection fluid to be sprayed into the room on the basis of a predefined required degree of purity; and
one or more nozzles each arranged to generate a jet and thereby spray the disinfection fluid into the room.

2. The disinfection unit according to claim 1 further comprising a mobile device configured to move the disinfection unit.

3. The disinfection unit according to claim 2, wherein the mobile device is integrated with the disinfection unit.

4. The disinfection unit according to claim 2, wherein the mobile device is electrically and mechanically connected to the disinfection unit.

5. The disinfection unit according to claim 1, further comprising a housing provided with a plurality of nozzles directed in different directions.

6. The disinfection unit according to claim 5, wherein the disinfection unit is configured to carry out a position-specific distribution of the disinfection fluid so that the disinfection fluid is distributed in dependency of position-specific height measurements from the detection unit.

7. The disinfection unit according to claim 1, wherein the disinfection unit is configured to carry out a position-specific distribution of the disinfection fluid so that the disinfection fluid is distributed in dependency of position-specific height measurements from the detection unit.

8. The disinfection unit according to claim 1, wherein the disinfection unit is configured to carry out a position-specific distribution of the disinfection fluid such that a volume of the disinfection fluid is distributed in dependency of one or more height measurements carried out by the detection unit while the disinfection unit is within 2 meters from an actual position.

9. The disinfection unit according to claim 1, wherein the control unit is arranged and configured to receive signals from one or more external device(s) and to control activity of the disinfection unit based on the signals.

10. The disinfection unit according to claim 9, wherein the control unit is configured to stop the disinfection unit if the signals indicate that:
a) a person is in the room to be disinfected; or
b) the ventilation system is turned on.

11. The disinfection unit according to claim 9, wherein the external device(s) is/are selected from the group consisting of a reader box, a motion sensor, an infrared sensor, a door sensor, a ventilation device, and combinations thereof.

12. A disinfection system comprising:
a disinfection unit according to claim 1;
a ventilation system; and
one or more communication unit(s) arranged and configured to detect activity of the ventilation system,
wherein the disinfection system is configured to control activity of the disinfection unit based on the detected activity of the ventilation system.

13. The disinfection system according to claim 12, further comprising a reader box arranged and configured to read a key member used by persons entering or leaving the building in which the room is located, wherein the one or more communication unit(s) is/are arranged and configured to detect activity of the reader box, wherein the disinfection system is configured to control activity of the disinfection unit based on the detected activity of the reader box.

14. A disinfection system comprising:
a disinfection unit according to claim 1;
a reader box arranged and configured to read a key member used by persons entering or leaving the building in which the room is located; and
one or more communication unit(s) arranged and configured to detect activity of the reader box,
wherein the disinfection system is configured to control activity of the disinfection unit based on the detected activity of the reader box.

15. The disinfection system according to claim 14, further comprising a ventilation system, wherein the one or more communication unit(s) is/are arranged and configured to detect activity of the ventilation system, wherein the disinfection system is configured to control activity of the disinfection unit based on the detected activity of the ventilation system.

16. A method for disinfecting a room having a ceiling, the method comprising:
providing the disinfection unit of claim 1;
detecting a height of the room using the detection unit of the disinfection unit;
calculating a quantity of disinfection fluid to be sprayed into the room on the basis of a predefined required degree of purity using the control unit;
using the position determination device to determine the position of the disinfection unit within the room; and
spraying the quantity of the disinfection fluid into the room through the one or more nozzles of the disinfection unit.

17. The method according to claim 16 further comprising moving the disinfection unit with a mobile device that is electrically and mechanically connected to the disinfection unit.

18. The method according to claim 16, further comprising controlling activity of the disinfection unit based on signals received from one or more external devices.

19. The method according to claim 18, wherein the step of controlling activity comprises stopping the disinfection unit if the signals indicate that:
a) a person is in the room to be disinfected; or
b) the ventilation system is turned on.

20. The method according to claim 16, wherein the step of spraying comprises carrying out a position-specific distribution of the disinfection fluid so that the disinfection fluid is distributed in dependency of position-specific height measurements from the detection unit.
